(19) United States
(12) Patent Application Publication
(10) Pub. No.: US 2012/0071935 A1
(43) Pub. Date: Mar. 22, 2012

(54) METHODS AND DEVICES FOR TREATING HALLUX VALGUS

(75) Inventors: Peter T. Keith, Lanesboro, MN (US); Nick T. Mourlas, Mountain View, CA (US)

(73) Assignee: Tarsus Medical Inc., Menlo Park, CA (US)

(21) Appl. No.: 13/222,631

(22) Filed: Aug. 31, 2011

Related U.S. Application Data
(63) Continuation-in-part of application No. 12/371,354, filed on Feb. 13, 2009.
(60) Provisional application No. 61/378,534, filed on Aug. 31, 2010.

(51) Int. Cl. A61B 17/84

(52) U.S. Cl. 606/328

(57) ABSTRACT

The various embodiments disclosed herein relate to methods and devices for treating a bone deformity such as hallux valgus. The various methods and devices relate to first and second anchors that are configured to be anchored in adjacent bones such that the two anchors, and thus the two bones, can be urged toward each other to treat the bone deformity.

### CROSS-REFERENCE TO RELATED APPLICATION

This application claims priority to Provisional Application No. 61/378,534, filed Aug. 31, 2010. This application is also a continuation-in-part of U.S. patent application Ser. No. 12/371,354, filed Feb. 13, 2009. Both of these applications are hereby incorporated herein by reference in their entireties.

### FIELD OF THE INVENTION

The various embodiments disclosed herein relate to methods and devices for treating a structural bone and joint deformity. More specifically, certain embodiments relate to systems and methods for correcting such deformity, including hallux valgus.

### BACKGROUND OF THE INVENTION

Hallux valgus deformities in the human foot typically relate to at least one of two conditions: a deviated position of the great toe where the great toe leans in towards the second toe (also referred to as the “hallux valgus angle” or “HV angle” as described below), and a deviation in the angle between the first and second metatarsal bones of the foot (also referred to as the “intermetatarsal angle” or “IM angle”). The most commonly used medical terms associated with these deformities are “hallux valgus” and “hallux abducto valgus,” where “hallux” refers to the great toe, “valgus” refers to the deformity in the frontal plane of an abnormal rotation of the great toe, and “abducto” refers to the abnormal slant or leaning of the great toe towards the second toe, as shown in FIG. 1. Hallux valgus is also commonly referred to in layman's terminology as a “bunion,” but the term “bunion” is more properly understood as the pathological bump, bony eminence, callous, and/or inflammation on the side of the great toe joint associated with either a bursal sac or structural deformity of the great toe as described above.

Various treatments for hallux valgus and/or bunions exist. Various surgical procedures may address some combination of removing the abnormal bony enlargement of the first metatarsal bone, realigning portions of the first metatarsal bone relative to the adjacent metatarsal bone via an osteotomy, straightening the first metatarsal bone relative to the adjacent toes through manipulations of the joint capsule, realigning the cartilagenous surfaces of the great toe joint, and/or repositioning the sesamoid bones beneath the first metatarsal bone. Other treatments can include bunion pads and external splints. All of these known treatments have shortcomings in either effectiveness (pads and splints) or invasiveness (the surgical procedures). With respect to the existing surgical procedures, the vast majority require an osteotomy for realignment of portions of the first metatarsal bone, which leads to long recovery and the need for patients to wear a cast or surgical boot for weeks following the operation, as well as the need to “stage” the procedure if both feet require surgical correction, i.e., treating one foot in a first surgery and then the other in a subsequent second surgery. Further, the surgical patients are left with a significant scar and poor cosmesis. In addition, studies have highlighted that as many as 30% of bunion surgery patients are unhappy with the result and nearly 10% have post-surgical complications. Finally, the surgical procedures are costly, requiring anesthesia, a lengthy operating time, and multiple trained medical staff.

Thus, there is a need in the art for improved methods and devices for treating hallux valgus.

### BRIEF SUMMARY OF THE INVENTION

Discussed herein are various treatment devices for treating syndesmosis injuries.

In Example 1, a method of treating hallux valgus comprises forming an incision in patient's foot, positioning a first bone anchor through the incision and into the first metatarsal bone, and positioning a second bone anchor through the incision and into the second metatarsal bone. In this example, the first bone anchor is coupled to a distal end of a tether and the tether is slidably coupled to the second bone anchor. The example further comprises urging a portion of the tether proximal to the second bone anchor in a proximal direction, whereby a distance between the first and second bone anchors is reduced, and fixedly coupling the tether to the second bone anchor.

In Example 2, a method of treating hallux valgus comprises positioning a first bone anchor through an incision between a first metatarsal and a second metatarsal in a patient's foot and into the first metatarsal bone, and positioning a second bone anchor through the incision and into the second metatarsal bone. In this example, the first bone anchor comprises a first coupling component that is coupled to a distal end of a tether, and the second bone anchor comprises a second coupling component, wherein the tether is slidably disposed through the second coupling component. The example further comprises urging a portion of the tether proximal to the second bone anchor in a proximal direction, whereby the first bone anchor is urged toward the second bone anchor, and fixedly coupling the tether to the second bone anchor.

Example 3 relates to the method of treating hallux valgus according to Example 2, wherein the first and second coupling components are eyelets.

In Example 4, a method of treating hallux valgus comprises positioning a first anchor into a first metatarsal bone, positioning a second anchor into a second metatarsal bone, and securing a tether to the first and second anchors.

### BRIEF DESCRIPTION OF THE DRAWINGS

FIG. 1 is a schematic depiction of a foot exhibiting hallux valgus.

FIG. 2 is a schematic depiction of a device for treating hallux valgus, according to one embodiment.

FIG. 3 is a schematic depiction of a device for treating hallux valgus being implanted into a patient, according to one embodiment.

FIG. 4 is a schematic depiction of a device for treating hallux valgus being implanted into a patient, according to one embodiment.

FIG. 5 is a schematic depiction of a device for treating hallux valgus that has been implanted into a patient, according to one embodiment.

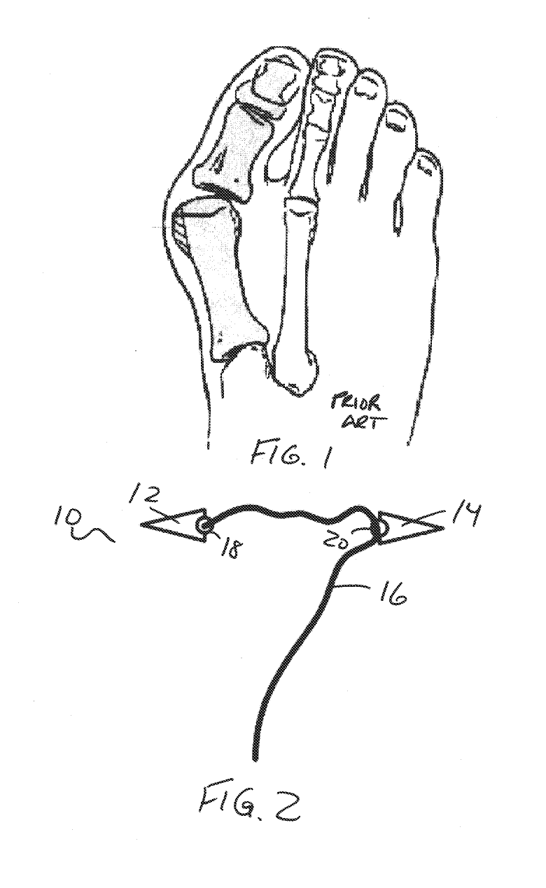

### DETAILED DESCRIPTION

Various embodiments disclosed herein relate to methods and devices for treating a bone deformity, such as, for example, hallux valgus (bunions). More specifically, various embodiments herein relate to bone deformity treatments using tension or connection systems and methods for anchoring or otherwise coupling metatarsal bones such as the first and second metatarsal bones. Some of the various device and method embodiments disclosed herein operate at least in part by anchoring or coupling to the heads of the first and second metatarsal bones. As such, various embodiments disclosed herein provide systems and methods for implantation of treatment devices and treatment of hallux valgus with reduced trauma and quicker recovery in comparison to known systems and treatments.

FIG. 2 depicts one embodiment of a device or system 10 that can be used to couple the first and second metatarsals of a foot. In this embodiment, the device 10 has a first anchor 12 and a second anchor 14 and a tether 16 that can couple the first and second anchors 12, 14. The tether 16 is tied or otherwise attached to an eyelet 18 on the first anchor 12 and further is slidably disposed through the eyelet 20 of the second anchor 14. Alternatively, the tether 16 can be attached to the eyelet 20 of the second anchor 14 and slidably disposed through the eyelet 18 of the first anchor 12. In a further alternative, the tether 16 can be coupled to the anchors 12, 14 in any known fashion so long as the device 10 has a configuration that allows the tether 16 to urge the two anchors 12, 14 closer together or otherwise reduce the distance between the two anchors 12, 14.

In the implementation depicted in FIG. 2, the anchors 12, 14 are illustrated generically, but could be pronged anchors such as the G2™ anchors which are commercially available from Depuy Mitek, Inc., which is based in Raynham, Mass. It is understood that the term “bone anchor” (or, alternatively, “anchor”), as used herein, is intended for purposes of this application to mean any component or device that can be used with any of the treatment device embodiments disclosed herein for anchoring or coupling such treatment devices to a bone. According to one embodiment, either or both of the anchors 12, 14 can be pronged bone anchors, externally-threaded anchors, harpoon type anchors, anchors with deployable arms, or any anchors or attachment components disclosed or claimed in any of U.S. application Ser. No. 12/371,354, filed on Feb. 13, 2009 and entitled “Methods and Devices for Treating Hallux Valgus,” U.S. application Ser. No. 12/567,314, filed on Sep. 25, 2009 and entitled “Methods and Devices for Treating a Structural Bone and Joint Deformity,” U.S. application Ser. No. 12/691,646, filed on Jan. 21, 2010 and entitled “Methods and Devices for Treating Hallux Valgus,” or U.S. application Ser. No. 12/793,429, filed on Jun. 3, 2010 and entitled “Methods and Devices for Treating Hallux Valgus,” all of which are hereby incorporated herein by reference in their entireties.

“Tether,” as used herein, is intended to mean any elongate component for use with medical devices such as suture, thread, a tube, or any other such material or device or combination thereof that can couple or be tensioned between two components such as anchors to treat bone deformations. The tether 16 in FIG. 2 can be a polyester suture line. Alternatively, the tether 16 is any flexible, suture-like material. In a further embodiment, the tether 16 is made of a material having longitudinal elasticity, such as, for example, polyurethane, Kraton, or a similar material. In yet another implementation, the tether 16 is made of a composite material. Further, the tether 16 can be any other tether or made of any other material as disclosed or claimed in any of U.S. application Ser. No. 12/371,354, filed on Feb. 13, 2009 and entitled “Methods and Devices for Treating Hallux Valgus,” U.S. application Ser. No. 12/567,314, filed on Sep. 25, 2009 and entitled “Methods and Devices for Treating a Structural Bone and Joint Deformity,” U.S. application Ser. No. 12/691,646, filed on Jan. 21, 2010 and entitled “Methods and Devices for Treating Hallux Valgus,” or U.S. application Ser. No. 12/793,429, filed on Jun. 3, 2010 and entitled “Methods and Devices for Treating Hallux Valgus,” all of which are incorporated by reference above.

It is understood that, in alternative embodiments, the tether 16 can be coupled to the anchors 12, 14 by components other than the eyelets 18, 20. In other words, the tether 16 can be coupled to the anchors 12, 14 by any known coupling or attachment components, so long as the device 10 can operate to urge the two metatarsals together. According to one embodiment, the anchors 12, 14 can have any attachment components disclosed or claimed in any of U.S. application Ser. No. 12/371,354, filed on Feb. 13, 2009 and entitled “Methods and Devices for Treating Hallux Valgus,” U.S. application Ser. No. 12/567,314, filed on Sep. 25, 2009 and entitled “Methods and Devices for Treating a Structural Bone and Joint Deformity,” U.S. application Ser. No. 12/691,646, filed on Jan. 21, 2010 and entitled “Methods and Devices for Treating Hallux Valgus,” or U.S. application Ser. No. 12/793,429, filed on Jun. 3, 2010 and entitled “Methods and Devices for Treating Hallux Valgus,” all of which are incorporated by reference above.

In use, according to one embodiment, a device can be implanted in the following fashion. As shown in FIG. 3, an incision 44 is made on the top of the foot between the first and second metatarsals 40, 42. A first anchor 48 of an implantable device 46 is inserted through the incision 44 and deployed into the first metatarsal 40 with the tether 52 coupled thereto. According to one embodiment, the anchor 48 is deployed into the head of the first metatarsal 40. That is, the anchor 48 is inserted, drilled, or otherwise positioned into the first metatarsal 40.

Next, the second anchor 50 is inserted through the incision 44 and deployed into the second metatarsal 42 with the tether 52 slidably coupled to the anchor 50. In one embodiment, the anchor 50 is deployed into the head of the second metatarsal 42. Alternatively, the anchors 48, 50 can be positioned anywhere along the metatarsals 40, 42.

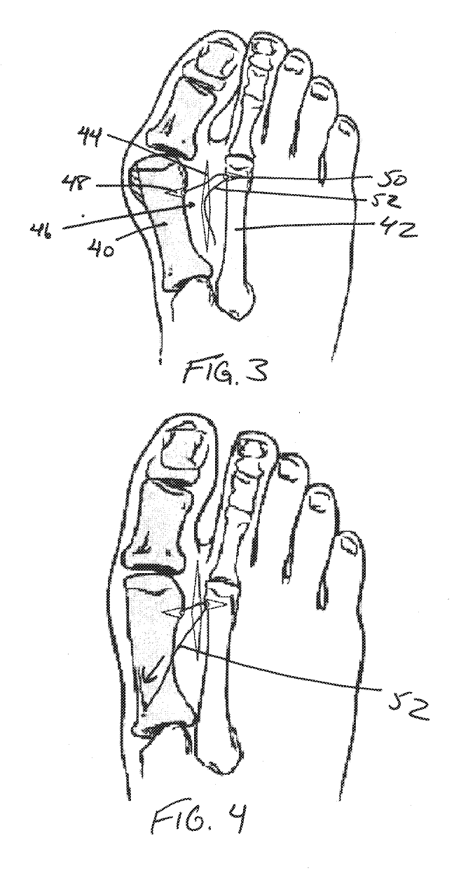

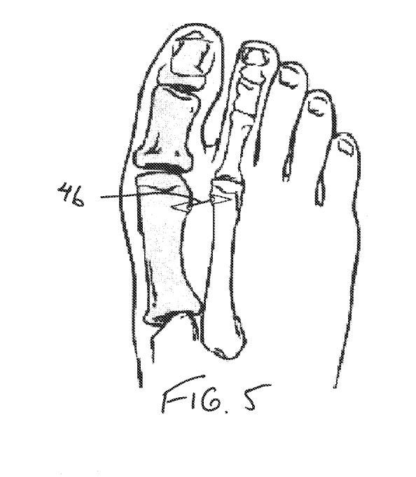

As shown in FIG. 4, once both of the anchors 48, 50 are positioned in the metatarsals 40, 42, the proximal end of the tether 52 can be pulled or otherwise urged away from the patient's foot (in the direction shown by the arrow in FIG. 4), thereby urging the first anchor 48 toward the second anchor 50, which thereby urges the first metatarsal 40 toward the second 42, as shown in FIG. 4. In this way, the amount of deviation exhibited by the first metatarsal 40 as shown in FIG. 3 is reduced.

FIG. 5 depicts the step in which the extraneous length of tether 52 extending from the second anchor 50 is removed. According to one embodiment, once the tether 52 is pulled as shown in FIG. 4 or the two metatarsals 40, 42 are otherwise move toward each other, the tether 52 is fixedly attached to the second anchor 50 and the excess tether 52 is removed. In one implementation, the tether 52 is tied to the anchor 50 with a knot and then the excess tether 52 is cut, such as with a scalpel. Alternatively, the tether 52 is attached to the anchor 50 and the excess tether 52 removed by any known methods. The resulting implanted device 46 is configured to maintain a maximum distance between the two metatarsals 40, 42, thereby providing treatment for hallux valgus.

Once the device 46 is in place and the excess tether 52 removed, the incision 44 can be closed by any method.

It is understood that each of the various device and method embodiments disclosed herein can be the sole treatment for the bone deformity. It is further understood that any of these embodiments could also be used in conjunction with any one or more of other known treatments, such as surgical removal of the eminence, surgical repositioning of the bones, surgical manipulation of the joint capsule (capsulorraphy), surgical removal of the underlying bunion, pads, splints, or any other treatment method or device.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

Although the present invention has been described with reference to preferred embodiments, persons skilled in the art will recognize that changes may be made in form and detail without departing from the spirit and scope of the invention.

What is claimed is:

1. A method of treating hallux valgus, comprising:
forming an incision in a patient's foot between a first metatarsal bone and a second metatarsal bone;
positioning a first bone anchor through the incision and into the first metatarsal bone, wherein the first bone anchor is coupled to a distal end of a tether;
positioning a second bone anchor through the incision and into the second metatarsal bone, wherein the tether is slidably coupled to the second bone anchor;
urging a portion of the tether proximal to the second bone anchor in a proximal direction, whereby a distance between the first and second bone anchors is reduced; and
fixedly coupling the tether to the second bone anchor.

2. A method of treating hallux valgus, comprising:
positioning a first bone anchor through an incision between a first metatarsal and a second metatarsal in a patient's foot and into the first metatarsal bone, wherein the first bone anchor comprises a first coupling component that is coupled to a distal end of a tether;
positioning a second bone anchor through the incision and into the second metatarsal bone, wherein the second bone anchor comprises a second coupling component, wherein the tether is slidably disposed through the second coupling component;
urging a portion of the tether proximal to the second bone anchor in a proximal direction, whereby the first bone anchor is urged toward the second bone anchor; and
fixedly coupling the tether to the second bone anchor.

3. The method of claim 2, wherein the first and second coupling components are eyelets.

4. A method of treating hallux valgus, comprising:
positioning a first anchor into a first metatarsal bone;
positioning a second anchor into a second metatarsal bone; and
securing a tether to the first and second anchors.
